(19) United States
(12) Patent Application Publication
(10) Pub. No.: US 2021/0382061 A1
(43) Pub. Date: Dec. 9, 2021

(54) MULTIPLEXED SINGLE-CELL ANALYSIS USING OPTICALLY-ENCODED RNA CAPTURE PARTICLES

(71) Applicant: THE GENERAL HOSPITAL CORPORATION, Boston, MA (US)

(72) Inventors: Seok-Hyun Yun, Cambridge, MA (US); Paul Dannenberg, Boston, MA (US)

(21) Appl. No.: 17/287,349

(22) PCT Filed: Oct. 22, 2019
(86) PCT No.: PCT/US19/57320

§ 371 Date: Apr. 21, 2021

Related U.S. Application Data
(60) Provisional application No. 62/748,849, filed on Oct. 22, 2018.

(51) Int. Cl. G01N 33/58; C12Q 1/6869

(52) U.S. Cl. CPC G01N 33/582; C12Q 1/6869; G01N 33/588

(57) ABSTRACT

An apparatus for capturing biological material. The apparatus includes: an optically readable capture particle (ORCP) including: one or more optically readable particles (ORPs) each including an optical barcode to identify the ORCP; and a plurality of biological capture sites associated with the one or more ORPs, each of the plurality of biological capture sites including a cellular barcode to identify the ORCP.

### CROSS-REFERENCE TO RELATED APPLICATIONS

This application claims the benefit of U.S. Provisional Patent Application Ser. No. 62/748,849 filed on Oct. 22, 2018, and entitled “Multiplexed Single-Cell Analysis Using Optically-Encoded RNA Capture Particles,” which is incorporated by reference herein in its entirety.

### BACKGROUND INFORMATION

Bead-based single cell RNA sequencing technologies (such as Dropseq, Indrops, Seqwell) allow users to rapidly profile the transcriptomes of thousands of individual cells. These methods work by marking the RNA transcripts originating from the same cell with a unique nucleotide-based cellular barcode. By using this barcode, it is possible to determine which profiled RNA transcripts originated from the same cell. However, these methods do not allow researchers to determine from which specific cell each transcript originated. Therefore, bead-based single cell RNA sequencing methods cannot be used to couple transcriptome information to any previously obtained single cell phenotypic data (e.g. cell size, protein expression etc.).

Thus, there is a need for improved single-cell RNA analysis tools.

### SUMMARY OF THE INVENTION

Disclosed herein are methods, systems, and apparatus for creating a large number (e.g. millions) of microparticles in solution, each of which emits light with unique spectral characteristics. These spectral characteristics, which are generated through the process of optical resonance, allow each particle (a resonator) to act as an optical barcode. In this disclosure, we discuss how the nucleotide-based cellular barcodes used in many single-cell RNA sequencing technologies could be incorporated onto these resonators to create collections of individual particles, each with their own cellular barcode and, simultaneously, their own optical barcode. The methodology we describe establishes a mapping between a set of optical barcodes and nucleotide-based cellular barcodes. By reading out these optical barcodes during the sequencing process, it is then possible to identify from which cell individual RNA transcripts originated.

Many commercially available high throughput single cell RNA sequencing methods can only determine which RNA transcripts came from the same cell. However, they are unable to tell from which individual cell the transcript originated. Therefore, any phenotypic characteristics of the cell cannot be associated with transcriptomic information obtained using these methods.

For example, if a researcher has 10,000 cells, and she measures the fluorescence intensity of a protein of interest in each of these cells, she can obtain quantitative data, reflective of the protein's expression levels in individual cells. However, if she then performs high throughput single cell sequencing using current, commercially available methods she will be able to deduce the transcriptomes of individual cells, but not which transcript is associated with a particular value of fluorescence intensity.

In this disclosure, we show how this challenge can be overcome by creating microparticles each of which possess a unique optical barcode. These barcodes can easily be read using a specific form of microscopy. Furthermore, we disclose how these microparticles can be adorned with nucleotide-based cellular barcodes in a way that establishes a mapping between each particle's optical and cellular barcode. The result is a particle that possesses a nucleotide barcode that can be read optically at speeds of one thousand barcode per second (kilohertz) or faster. We expect these particles to have broad application across the field of single cell analysis.

Thus in one embodiment the invention provides an apparatus for capturing biological material. The apparatus includes: an optically readable capture particle (ORCP) including: one or more optically readable particles (ORPs) each including an optical barcode to identify the ORCP; and a plurality of biological capture sites associated with the one or more ORPs, each of the plurality of biological capture sites including a cellular barcode to identify the ORCP.

In various embodiments of the apparatus, the one or more ORPs may comprise a resonator and gain medium.

In various other embodiments of the apparatus, the one or more ORPs may comprise a microsphere.

In certain other embodiments of the apparatus, the microsphere may be doped with the gain medium.

In still other embodiments of apparatus, the gain medium may include a fluorescent material.

In yet other embodiments of the apparatus, the fluorescent material may be at least one of a fluorescent dye, a quantum dot, or a protein.

In certain other embodiments of the apparatus, the microsphere may include a polystyrene microsphere.

In various other embodiments of the apparatus, the microsphere may have a diameter of at least 3 μm.

In yet other embodiments of the apparatus, the optical barcode may include an emission spectrum having at least one peak.

In still other embodiments of the apparatus, the ORCP may include a plurality of gain media and a plurality of resonators, wherein each of the plurality of gain media includes a different emission spectrum.

In certain other embodiments of the apparatus, the one or more ORPs may include a semiconductor particle which includes the resonator and the gain medium.

In yet other embodiments of the apparatus, the semiconductor particle may be contained within a transparent coating.

In certain other embodiments of the apparatus, the one or more ORPs may include a plurality of semiconductor particles contained within the transparent coating.

In still other embodiments of the apparatus, each of the plurality of semiconductor particles may include a gain medium having a different emission spectrum from the other of the plurality of semiconductor particles.

In various other embodiments of the apparatus, each of the plurality of biological capture sites may include a nucleotide strand configured to capture RNA.

In certain other embodiments of the apparatus, the nucleotide strand may include an oligonucleotide cellular barcode sequence.

In another embodiment the invention provides a method of capturing biological material, including: combining an ORCP and a cell within an aqueous environment, the ORCP comprising: one or more optically readable particles (ORPs) each comprising an optical barcode to identify the ORCP, and a plurality of biological capture sites coupled with the one or more ORPs, each of the plurality of biological capture sites including a cellular barcode to identify the ORCP, and each of the plurality of biological capture sites including a capture site for capture of biological material; reading the optical barcode of the ORCP; identifying a phenotypic property of the cell; capturing contents of the cell such that they interact with the plurality of biological capture sites of the ORCP; and processing the ORCP to identify the contents of the cell associated with the plurality of biological capture sites.

In various embodiments of the method, the one or more ORPs may include a resonator and a gain medium.

In certain embodiments of the method, the one or more ORPs may include a microsphere.

In yet other embodiments of the method, the microsphere may be doped with the gain medium.

In still other embodiments of the method, the gain medium may include a fluorescent material including at least one of a fluorescent dye, a quantum dot, or a protein.

In various embodiments of the method, the microsphere may include a polystyrene microsphere having a diameter of at least 3 μm.

In certain embodiments of the method, the one or more ORCP may include a semiconductor particle.

In various embodiments of the method, the semiconductor particle may include the resonator and the gain medium.

In yet other embodiments of the method, the semiconductor particle may be contained within a transparent coating.

In various other embodiments of the method, the ORCP may include a plurality of semiconductor particles contained within the transparent coating.

In yet other embodiments of the method, each of the plurality of semiconductor particles may include a gain medium having a different emission spectrum from the other of the plurality of semiconductor particles.

In certain embodiments of the method, the optical barcode may include an emission spectrum having at least one peak.

In some embodiments of the method, each of the plurality of biological capture sites may include a nucleotide strand configured to capture RNA.

In various embodiments of the method, the nucleotide strand may include an oligonucleotide cellular barcode sequence.

In certain embodiments of the method, combining an ORCP and a cell within an aqueous environment may further include: combining the ORCP and the cell within an aqueous droplet that is immersed in oil.

In some embodiments of the method, processing the ORCP to identify the contents of the cell associated with the plurality of biological capture sites may further include: identifying the cellular barcode associated with the plurality of biological capture sites.

In still other embodiments of the method, reading the optical barcode of the ORCP may further include: directing a light source at the ORCP; detecting a return light spectrum emitted by the ORCP based on directing the light source at the ORCP; and determining the optical barcode by analyzing the return light spectrum to identify at least one peak within the return light spectrum.

In yet other embodiments of the method, identifying a phenotypic property of the cell may further include: identifying an observable property related to the cell relating to at least one of protein quantification, cell cycle information, gene expression, cell location, cell mass, and intercellular interactions.

In certain other embodiments of the method, identifying a phenotypic property of the cell may further include: directing a fluorescent excitation source at the cell; detecting fluorescent emission light from a fluorescent reporter associated with the cell based on directing the fluorescent excitation source at the cell; and identifying the phenotypic property of the cell based on detecting the fluorescent emission light.

In further embodiments of the method, combining an ORCP and a cell within an aqueous environment may further include: providing a plurality of ORCPs on a surface; placing the cell adjacent the plurality of ORCPs; identifying a location of each of the plurality of ORCPs relative to the cell by reading the optical barcode of each of the plurality of ORCPs; identifying the phenotypic property of the cell; releasing cellular contents from the cell; and processing each of the plurality of ORCPs to identify the contents of the cell associated with the plurality of biological capture sites associated with each of the plurality of ORCPs.

In still another embodiment the invention provides an apparatus for capturing biological material, including: a plurality of optically readable capture particles (ORCPs), each ORCP including a plurality of optically readable particles (ORPs) and a plurality of biological capture sites, each of the plurality of ORPs including an optical barcode to identify the ORCP; and a plurality of biological capture sites coupled to each of the plurality of ORPs, each of the plurality of biological capture sites including a cellular barcode to identify the biological capture site.

In yet another embodiment the invention provides an apparatus for capturing biological material including an optically readable capture particle (ORCP) including: a plurality of optically readable particles (ORPs) and a plurality of oligonucleotide-based cellular barcodes in which an association has been established between an optical barcode of the plurality of ORPs and the oligonucleotide-based cellular barcode, wherein: knowledge of a sequence of the oligonucleotide-based cellular barcode enables a determination of the optical barcode of the plurality of ORPs, or knowledge of the optical barcode of the plurality of ORPs enables a determination of the oligonucleotide-based cellular barcode.

In other embodiments of the apparatus, each of the plurality of ORCPs may include a plurality of resonators wherein each resonator has a different size, and each of the plurality of ORPs may have a different optical barcode based on the different size.

In still other embodiments of the apparatus, each of the plurality of ORPs may include a plurality of resonators and a plurality of gain media and has a same gain medium.

In certain other embodiments of the apparatus, the plurality of ORPs and the plurality of biological capture sites may be embedded within a hydrogel bead.

In various other embodiments of the apparatus, the plurality of ORPs may include a resonator and gain medium.

In yet other embodiments of the apparatus, the plurality of ORPs and the plurality of biological capture sites may be embedded within the hydrogel bead using a microfluidic device, and the microfluidic device may form an emulsion of aqueous based droplets containing the plurality of ORPs and the plurality of biological capture sites, and the droplets may be subsequently cured into hydrogel beads.

In various embodiments, a method of associating an optical barcode with a cellular barcode for an apparatus for capturing biological material may include: forming the cellular barcode through a plurality of rounds of split-and-pool synthesis procedures wherein a known subsection of the cellular barcode associated with an ORP of the one or more ORPs is added during each round; and recording an identity of the optical barcode of the one or more ORPs of the ORCP during each of the plurality of rounds and associating the identity with the added subsection of the cellular barcode.

The foregoing and other aspects and advantages of the invention will appear from the following description. In the description, reference is made to the accompanying drawings which form a part hereof, and in which there is shown by way of illustration preferred embodiments of the invention. Such embodiments do not necessarily represent the full scope of the invention, however, and reference is made therefore to the claims herein for interpreting the scope of the invention.

### BRIEF DESCRIPTION OF THE DRAWINGS

Further objects, features and advantages of the present disclosure will become apparent from the following detailed description taken in conjunction with the accompanying figures showing illustrative embodiments of the present disclosure, in which:

FIG. 1 shows an exemplary optically readable capture particle (ORCP).

FIGS. 2A-2D show exemplary spectral patterns (barcodes) of optical resonators.

FIGS. 3A-3C show examples of optical resonators that could be used as part of an ORCP.

FIG. 4 shows a simplified overview of an exemplary ORCP fabrication process.

FIG. 5A shows a simplified setup to perform spectral flow cytometry. Such a system can be used to keep track of the different ORCPs during fabrication.

FIG. 5B shows an exemplary optical system that is used to both pump the laser particle/RNA capture resonator and record its spectral emission.

FIG. 6 shows how ORCPs can be used to couple analysis from imaging cytometry with single cell sequencing data.

FIG. 7 shows a simplified setup of a system that can perform combined flow cytometry with single cell sequencing.

FIG. 8 shows how ORCPs can be used to perform in situ sequencing of tissues, allowing the spatial distribution of RNA molecules to be deduced at a spatial resolution below that of a single cell.

FIG. 9 shows an example of a system for capturing and analyzing biological material in accordance with some embodiments of the disclosed subject matter.

FIG. 10 shows an example of hardware that can be used to implement a computing device and server in accordance with some embodiments of the disclosed subject matter.

FIG. 11A shows an example of ORCP beads comprising polystyrene microsphere ORPs.

FIG. 11B shows the spectral characteristics or the optical barcode of the polystyrene microsphere ORPs.

FIG. 12 shows an embodiment of a microfluidic process used to form the ORCP in FIG. 11A.

### DETAILED DESCRIPTION OF EXEMPLARY EMBODIMENTS

Complex tissues exhibit an enormous amount of phenotypic heterogeneity. Historically, these individual cells were categorized into groups based on only a few observable properties (e.g. size, shape, presence of stainable markers). Recently, the advent of single cell nucleic acid sequencing technologies has made it possible to quantitatively determine the transcriptome of individual cells, allowing researchers to better appreciate these intercellular differences. Such analysis has become fundamental to our understanding of the signatures of disease at the single cell level.

One high throughput method to perform single cell RNA sequencing involves the co-compartmentalization of individual cells with individual RNA capture microbeads. In one representative design (WO 2015/164212) each of these beads is lined with an RNA capture strand including a PCR amplification region, an oligo(dT) region for mRNA capture, a unique molecular identifier (to calibrate readouts for amplification noise), and a cellular barcode including a specific nucleotide sequence that is designed to be unique to each bead. Ideally there are a large number (e.g. at least several thousand) of beads each with different cellular barcode sequences. In many high throughput, single cell RNA sequencing protocols, each bead is randomly compartmentalized with just a single cell. The cell is then lysed and its RNA is captured by the RNA capture strands attached to the bead. Reverse transcription is then performed on the captured RNA, forming a cDNA library of the captured RNA. All beads are then pooled for in vitro amplification and high throughput sequencing.

Following sequencing analysis, it is possible to determine which RNA transcripts originated from the same cell by analyzing the cellular barcode sequence that was transferred, by reverse transcription, from the RNA capture strand to the cDNA. Unfortunately, while this tells us which transcripts originated from the same cell, it does not tell us which particular cell it came from, since the capture beads were randomly assigned to each cell. This means that current bead-based high throughput RNA sequencing technology does not allow the obtained single transcriptome analysis to be coupled to previously collected data regarding each cell's phenotype (e.g. size, protein expression levels, spatial location etc.). Others have recognized and stated this as a clear limitation of this family of methods (e.g. Zilionis et al. Nature Protocols 12, 44-73, 2017). Overcoming this limitation would allow researchers to couple genotype with phenotype at the single cell level.

One possible solution is to optically encode each RNA capture bead in such a way that the cellular barcode nucleotide sequence can be associated with the optical emission of each bead. Such a method would map each permutation of cellular barcode nucleotides to a unique optical property of the bead, essentially allowing the nucleotide-based cellular barcode sequence to be read optically. Any data obtained from subsequent sequencing could then be associated with the optical property of the bead, which in turn could be linked to phenotypic traits of the sequenced cell.

A potential optical encoding method would be to label each bead separately by using different fluorescent dyes at a range of concentrations so as to modulate their emission spectra in both intensity and center wavelength. However, commercial fluorescence-based systems that rely on this method can create only a few hundred unique optical barcodes; this number is too few to reliably perform high throughput sequencing, which often requires at least several thousand RNA capture beads. This fundamental problem stems from the fact that dyes, fluorescent probes, and even quantum dots have broad emission spectra, making unique optical identification in an unambiguous manner a challenging problem, due to spectral overlap.

Unlike the relatively broadband emission spectra of the aforementioned molecules, an optical resonator, when coupled with an appropriate gain medium, generally has an emission spectrum with a narrow full width at half maximum in the spectral domain, making it ideal for applications in which significant multiplexing is needed. Furthermore, the peak emission wavelength(s) can be coarsely tuned by varying the gain medium or finely tuned by making slight alterations to the size of the cavity responsible for the optical resonance, e.g. while using the same gain medium.

In this patent description, we introduce a method to fabricate RNA capture particles in such a way that their cellular barcode sequence can be mapped to the particle's optical emission spectrum in an unambiguous manner. This allows the nucleotide-based cellular barcode to be deduced by determining the particle's optical emission spectrum, essentially creating an optically readable capture particle (ORCP). In order to achieve a large number of unique emission spectra, we propose either physically coupling a miniature optical resonator along with a gain medium to each RNA capture particle, or using the resonator itself as the RNA capture particle.

An ORCP may include two functional components that are combined into a single physical entity. The first component is generally an RNA capture particle such as those described in WO 2015/164212 and WO 2016/040476. The second component is one or more optically readable particles (ORPs). One embodiment of an optically readable particle includes a micron-scale resonator, which, when optically pumped by an external light source, emits a unique spectral signature that can be measured by a spectrometer. The combination of these two functional parts possesses an unambiguous oligonucleotide cellular barcode as well as an unambiguous optical barcode. Embodiments of the disclosed invention are applicable to particle- or bead-based assays in which cellular material is captured on individual beads. While one particular application includes mapping of a single cell's transcriptome to prior phenotypic information regarding that same cell, in various embodiments the ORCPs may also be used with multiplexed bead-based assays to alternative single-cell profiling platforms such as DNA sequencing, ATAC-seq, and ChIP-seq.

In various embodiments, the invention includes RNA capture microparticles containing both a nucleotide-based cellular barcode and an optical barcode, split-and-pool tools to fabricate such optically-encoded, RNA-capture microparticles, microscopy systems designed to read out the optical barcodes of the capture particles, and/or single cell sequencing apparatus based on optical barcoding readout.

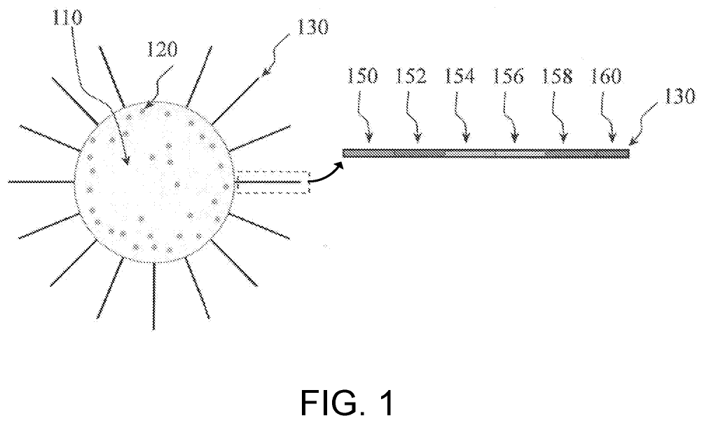

FIG. 1 shows an embodiment of an ORCP, in which a miniature resonator 110 containing fluorescent emitters 120 has been coupled to RNA capture strands 130 on its surface. The resonator particle acts as a unique optical barcode while the RNA capture strands attached to the resonator all have the same unique cellular barcode sequence. Different ORCPs have different cellular barcode sequences as well as different optical barcodes. Typically, each RNA capture strand 130 (e.g. such as those disclosed in WO 2015/164212 and WO 2016/040476) includes a cleavable region 150 that allows the strands to be separated from the particle, a primer region for polymerase chain reaction (PCR) 154, a promotor region(s) 152 for subsequent amplification, an oligonucleotide DNA cell barcode 156, a unique molecular identifier 158 to allow for some normalization of the effects of amplification, and a poly(dT) region 160 to allow for mRNA capture. In various embodiments, other molecular functional units may be included.

In one embodiment, a resonator particle may be formed from a polystyrene microsphere 120 (the cavity) doped with a fluorescent material such as a fluorescent dye, quantum dots, or protein (the gain medium 130). In each case, the gain medium should have an absorption spectrum spanning wavelengths over which the medium will absorb excitation light. Similarly, it should possess an optical emission spectrum. This encompasses a significant number of materials. Specific examples of fluorescent dyes that could act as a gain media include, but are not limited to, fluorescein isothiocyanate and tetramethylrhodamine. Examples of potential quantum dot gain media include, but are not limited to, PbS quantum dots, CsPbBr₃, CH₃NH₃PbCl₃. Examples of protein gain media include, but are not limited, to yellow fluorescent protein and green fluorescent protein (GFP). Typically, the gain medium should be chosen so as to minimize spectral crosstalk with other fluorescent sources that might be used during analysis. For example, if sequencing data is to be obtained from GFP expressing cells, a GFP-based gain medium would be less suitable for cellular identification since optical spillover from the ORCP could influence measurement of the cells' native GFP expression. To prevent ORCP emission spectra interfering with most common cell dyes, most versatility is afforded by using an optical gain medium for the ORCP that is active in the infrared. However, the relatively high cost to performance ratio of detectors at this wavelength make visible gain media more inviting to use when possible. In general, a single resonator will be coupled to a single gain medium. However, the spectral signature of an ORCP that allows it to be uniquely identifiable can provide additional multiplexing ability if it consists of multiple resonators coupled to multiple gain media.

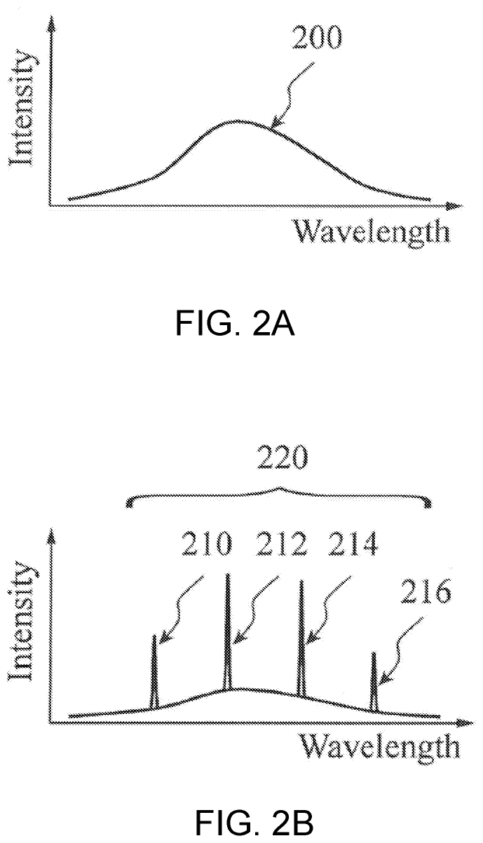

Normally, optical illumination of such a particle by an external light source would produce broad spectral, fluorescence emission, as shown in an exemplary fluorescence emission spectrum 200 in FIG. 2A. However, when the diameter of the microsphere is sufficiently large, typically >3 μm, pumping with sufficient power can lead to peaks in the emission spectrum of such a particle. The solution of Maxwell's equations in even the relatively simple case of a microsphere resonator can result in complicated expressions that describe the emission characteristics of the resonator's optical modes. A straightforward, approximate solution (Gorodetsky, Fomin, IEEE J. Sel. Topics Quantum Electron. 12, 33-39 2006) however, is known which describe the resonant wavenumbers k as

$${n}_{s} ka = l - {{α}_{q} ( \frac{l}{2} )}^{1 \text{/} 3} + \frac{1}{2} \frac{χ {n}_{r}}{\sqrt{{n}_{r}^{2} - 1}} + \frac{3 {α}_{q}^{2}}{20} {( \frac{l}{2} )}^{- 1 \text{/} 3} - \frac{{α}_{q}}{12} ( 1 + \frac{2 χ {n}_{r} ( 2 {χ}^{2} - 3 {n}_{r}^{2} )}{{( {n}_{r}^{2} - 1 )}^{3 \text{/} 2}} ) {( \frac{l}{2} )}^{- 2 \text{/} 3} ,$$

where the corresponding wavelength is given 2π/k, and l is the polar mode number, a is the microsphere radius, nₛ and nᵣ are the refractive indices of the microsphere and the surrounding medium respectively, a_{w} are the q-th zeroes of the Airy function with q denoting the possible radial modes. χ=1 for TE modes and χ=1/nᵣ for TM modes.

One of the advantages of using resonant wavelength based barcoding strategies is that it maps discrete (and therefore simple to detect and analyze) values of emission wavelengths to variables n and a that can be continuously varied to generate a diverse set of barcodes. Nevertheless, given a particular resonator and gain material, there are limitations on its size that render it useful for barcoding. One theoretical upper bound on resonator size is the point at which the free spectral range (FSR), which scales approximately as,

$$FSR ∼ \frac{{n}_{s} a}{{l}^{2}} ,$$

becomes too small to be able to distinguish separate peaks, which happens when the spacing is on the order of the emission linewidth. This linewidth is governed by resonator loss due to outcoupled radiation and, when laser emission is occurring, by optical gain generated by the gain medium. While there exist theoretical expressions that describe this linewidth, in practice it is best measured empirically since resonator loss can be significantly affected by scattering and absorption by inherent features of the material used. For a polystyrene microsphere of diameter approximately 10 μm, the energy required to observe a spectrum with sufficient signal to noise ratio of peaks is on the order of 1-10 nJ. FIG. 2B shows an exemplary spectrum including broadband fluorescence background and four resonance peaks 210, 212, 214, and 216. These peaks are caused by resonance of light trapped within the polystyrene cavity and are highly sensitive to the precise size of the microsphere. The center wavelengths of individual resonance peaks and the spectral differences among them can easily be determined using a spectrometer. The spectral properties uniquely characterize the resonance mode structure 220, which can serve as an optical barcode of the microsphere. A different microsphere with a different diameter exhibits a different mode structure 230 and therefore can be differentiated from other microspheres (FIG. 2D).

This behavior is observed because the microsphere acts as an optical resonator: polystyrene's relatively high refractive index (˜1.6) confines light within its interior, causing more photons, produced by stimulated emission, to build up within specific optical modes, which are known as whispering gallery modes. The resulting emission from the resonator includes one or more spectrally narrow peaks whose center wavelength is determined by the dimensions of the polystyrene microsphere. Under certain conditions, this resonance phenomenon can even lead to lasing from the microresonator. FIG. 2C shows an exemplary lasing spectrum, in which typically one resonance mode 240 or a few resonance modes turn into coherent stimulated emission. Ideally the fluorescent emitters 120 are preferably distributed along the surface so that broadband fluorescence background is minimized, and the resonance peaks are maximized. Besides polystyrene, other transparent polymers (e.g. melamine resin), glasses (e.g. fused silica, BaTiO₃), and crystals (e.g. diamond, ZnO) may be used as long as their refractive index is larger than the refractive index of the surrounding medium.

A polydisperse collection of such microspheres containing fluorescent emitters may be used as a collection of unique optical barcodes, where the barcode of each microsphere is determined in part by a size (e.g. radius) of the microsphere. Using a variety of different gain media along with microspheres of different sizes, a large number of unique barcodes can be formed, each of which can be read by optically pumping the particle and analyzing its spectral output. Our work has shown that a polydisperse set of beads in a size range of 8 μm to 12 μm and a single fluorescent dye can result in approximately 2,000 distinguishable optical emission spectra (Humar et al., Lab Chip, 2017, 17, 2777). Therefore, by combining multiple particles together, in various embodiments it is possible to form many millions of unique optical barcodes (for example, with 3 different resonators joined together, this would permit formation of C(2000, 3)≈10⁹ different optical barcodes).

In another embodiment, a set of direct-bandgap semiconductor resonator particles with polydisperse sizes are used. In this embodiment, the semiconductor material itself serves as both the gain medium and the microresonator. Interband transitions within the semiconductor may lead to optical gain, and resonance may occur by confinement within the semiconductor both due to its relatively high refractive index (typically >2.5) and its geometric shape (e.g. toroidal, discoid, cuboid, spherical etc.). Suitable semiconductor materials include III-V groups such as InAlGaAs, InGaAsP, and GaN, II-VI groups, and organic semiconductors etc. A polydisperse set of resonator particles can be fabricated by lithographic means (e.g. optical lithography, electron beam lithography, interference lithography etc.) and etched using standard semiconductor fabrication processes. One advantage of many semiconductor materials is their relatively high refractive index, which can allow for the production of narrow peaks in their emission spectra even at diameters smaller than a few microns and thicknesses of a few hundreds of nanometers in a microdisk geometry. Optionally, a transparent coating of material with refractive index lower than that of the semiconductor material can be used to prevent coupling of the optical modes of the particle to the surrounding environment (including other particles). An example of such a coating would be SiO₂ although other materials such as Si₃N₄ are also possible.

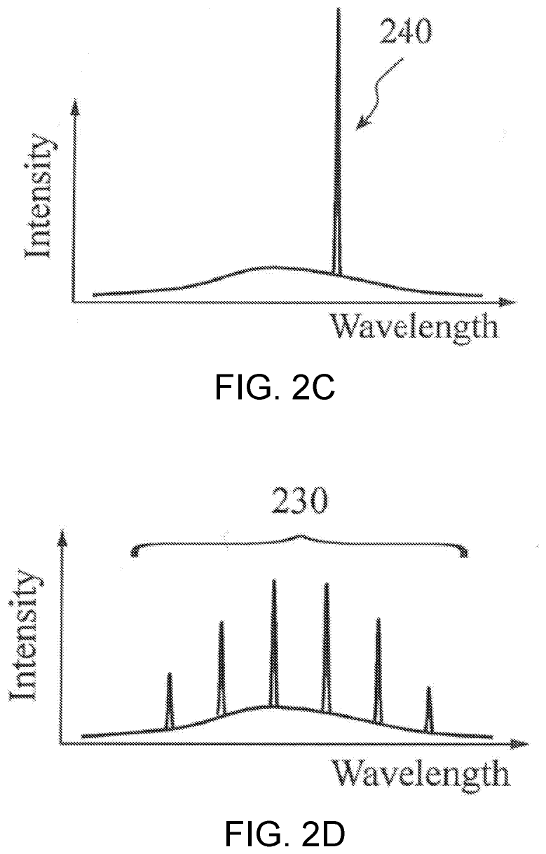

FIGS. 3A-3C show several possible embodiments of exemplary optical resonators for ORCPs. FIG. 3A shows a semiconductor disk resonator particle 300 (e.g. InAlGaAs) coated with a thin layer of SiO₂ 310 to form a coated semiconductor resonator/laser particle 320. The particle 320 is coated with RNA capture strands including DNA barcodes 330, similar to the RNA capture strands 130 depicted in FIG. 1A. FIG. 3B shows two semiconductor resonator particles, 300 and 340, that are joined together, for example by a monocrystalline layer of indium phosphide (350). The entire compound particle is then coated in SiO₂ layer 310. FIG. 3C shows multiples of such silica-coated semiconductor resonator particles (e.g. 320 and 360) encapsulated to form a single bead (370). The bead may be hydrogel or a polymer such as polystyrene. The bead is then coated with RNA capture strands 330.

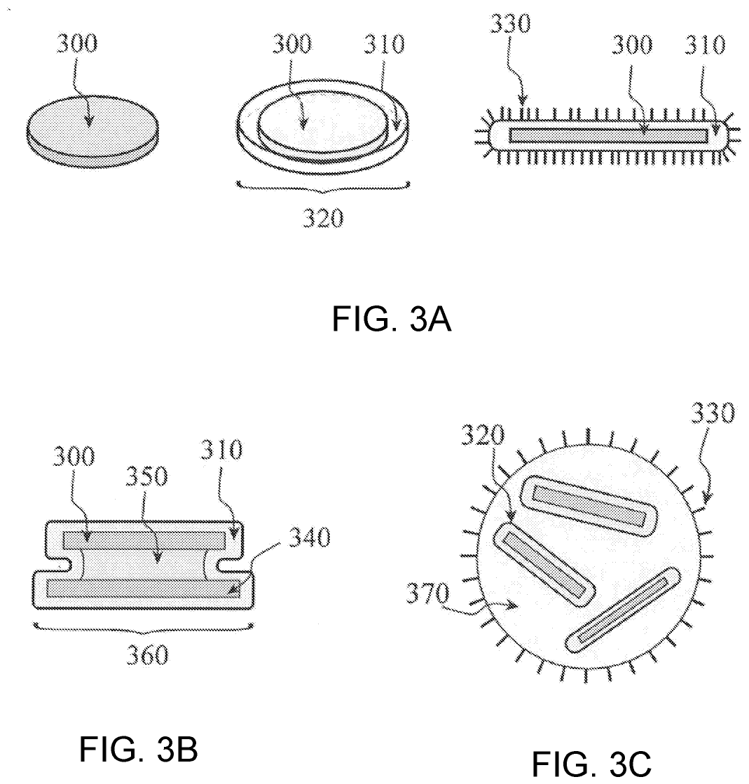

Attaching the RNA capture strands to the resonator particle can be performed by a variety of procedures. In some embodiments, a method similar to that disclosed in publication numbers WO 2015/164212 and WO 2016/040476 (which detail techniques to fabricate RNA capture particles) may be used. FIG. 4 shows a simplified overview of an embodiment of this technique. First, a large collection (typically >1,000) of optical particles are added to reagents containing DNA stubs, allowing them to couple to the laser particles. Each of these DNA stubs includes a cleavable region (e.g. for later release from the particle) along with a promotor region and an adaptor region. These two latter regions allow hybridization and strand elongation which allow the entire RNA capture strand to be synthesized from this original stub. Next, a modified ‘split and pool’ technique is performed on the optical particles, which have now been coupled to DNA stubs. This step produces a large collection of optical particles coupled to DNA stubs 400. Therefore, within this collection, a large number of different optical barcodes exist, denoted by the different colored particles.

In the next step 410, this collection is split into a number of subcollections 420, 422, and 424. For example, if the starting number of resonator particles lined with DNA stubs is 100,000, we might split this collection into 384 subcollections, each of which reside in the well of a 384 well plate. Following this splitting process, approximately 260 particles should reside within each well. To create an associative map between the optical barcode of an ORCP and its oligonucleotide cellular barcode, the splitting process, albeit random, should be performed in a known manner. Therefore, it is necessary to determine which particles were placed in which well.

During this splitting process 410, the optical barcodes are read out and recorded. Once the identities (defined by the emission spectra) of the particles which reside in each well have been determined, an oligonucleotide strand that represents part of the cellular barcode sequence, can be added to each well. A different oligonucleotide sequence, 430, 432, and 434, is added to each well. Next, these strands are joined to the particles' existing DNA stubs to form optical particles with DNA barcodes 440, 442, and 444. At this point, however, there would only be 384 different oligonucleotide barcodes. To achieve a more diverse set of cellular barcodes, these oligonucleotide-tagged resonator particles can then be pooled 450 into a large collection 460, and the procedure repeated 470 for splitting with optical barcode readout 410. After N rounds of this procedure, there would be approximately 384N different nucleotide barcodes. By performing spectral readout of each particle to determine into which well it was deposited, the optical barcode can be associated with the oligonucleotide-based cellular barcode. Therefore, by measuring the output emission spectrum from the RNA capture resonator particle, the nucleotide barcode can be deduced and vice versa.

Following this exemplary procedure, a large collection of ORCPs can be formed. Ideally, each of these particles will have a unique cellular barcode sequence as well as a unique optically readable spectral barcode. Furthermore, by performing a spectral readout of the optical barcode at each stage of the modified split-and-pool synthesis, a mapping between the cellular barcode and the optical barcode is known. In order to ensure there are no repeated optical barcodes, the apparatus may employ a sorting arrangement that is configured to remove any particles with duplicated spectra from the original collection of fabricated particles.

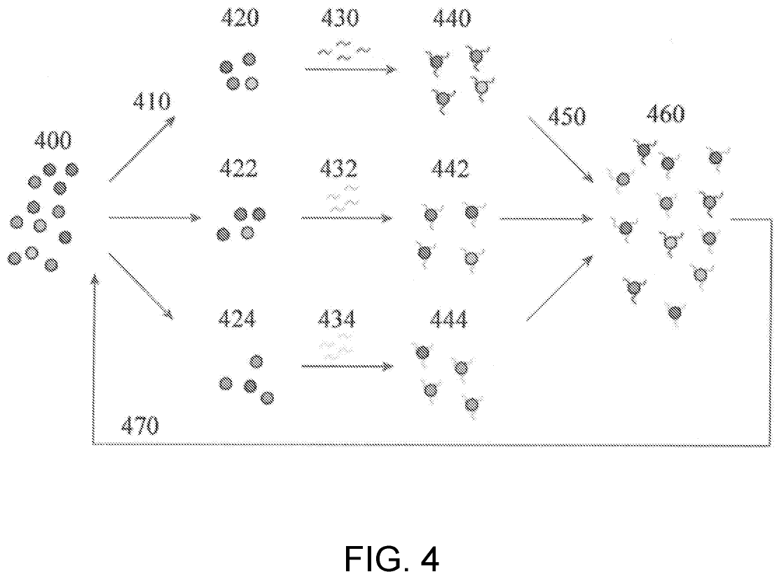

In one exemplary embodiment for the splitting and optical readout process 410, the splitting process could be performed by spectral flow cytometry. FIG. 5A shows an apparatus for flow-based optical readout. ORCP particles are loaded in a flow channel 500. An optical pump source 510 or multiple pump sources are employed. The pump sources may be pulsed lasers emitting nanosecond or longer pulses or they could be continuous wave-emitting lasers or light emitting diodes. The pump light is focused onto the flow channel 500. As a particle 520 passes by the focus, the pump excites the gain medium of the particle, and the output fluorescence or laser emission is directed to a spectrometer 530 through a dichroic mirror 540. The spectrometer 530 may be equipped with a diffraction grating and multichannel detector, such as a CCD or EMCCD to read out the optical barcode. Preferably, the spectrometer can resolve more than 500 spectral components with a resolution of <1 nm. At the end of the fluidic channel, the resonator particles are deposited into wells of the 384-well plate. Dispensing the output of the flow cytometer into multiple different wells could be achieved, for example, by actuating a series of valves to direct the flow into each well in some known order. Therefore, approximately the first 260 particles that are read are deposited into the first well, the next 260 into the next well etc. In this way, the complete set of particles is read, and each particle has its optical barcode recorded as well as the identity of the well that the particle is entering.

Alternatively, this optical barcode readout could be performed by spectral imaging cytometry. FIG. 5B illustrates another exemplary apparatus. In this embodiment, the original collection of approximately 100,000 ORCP particles is divided roughly evenly into each well of the 384-well plate 550. The sample 550 is placed atop a moving stage, which allows the system to interrogate the particles in different wells. After this, an optical microscope system locates each resonator particle by performing image analysis on a bright-field image using a light source 560 and camera 570, directs the output of an optical pump source 510 to each particle through the use of a pair of mirror galvanometers 580, and reads out its emission spectrum with a spectrometer 530, thereby recording its optical barcode. The optical pump source is chosen to provide excitation light with appropriately sufficient energy to generate observable optical signatures from the ORCPs. In the case of a WGM-based signature, the pump light therefore shares some spectral overlap with the absorption spectrum of the gain material in the ORCPs. While this could take the form of a lamp or LED, the relatively high energies needed mean the preferred embodiment would include a laser source (either continuous wave or pulsed).

### Hypothetical/Prophetic Examples of Uses of ORCPs

### Example 1: Associating Cytometry Data with Single Cell Sequencing

Optical reporters are used heavily in biological assays to quantitatively measure protein expression. While measurements of the reporters' output can be performed at a single cell level, for example, by using imaging cytometry or flow cytometry, it is not currently possible to associate the results of these assays with single cell RNA sequencing data obtained via high throughput, bead-based methods. The ORCPs disclosed in this invention offer a way to make this association.

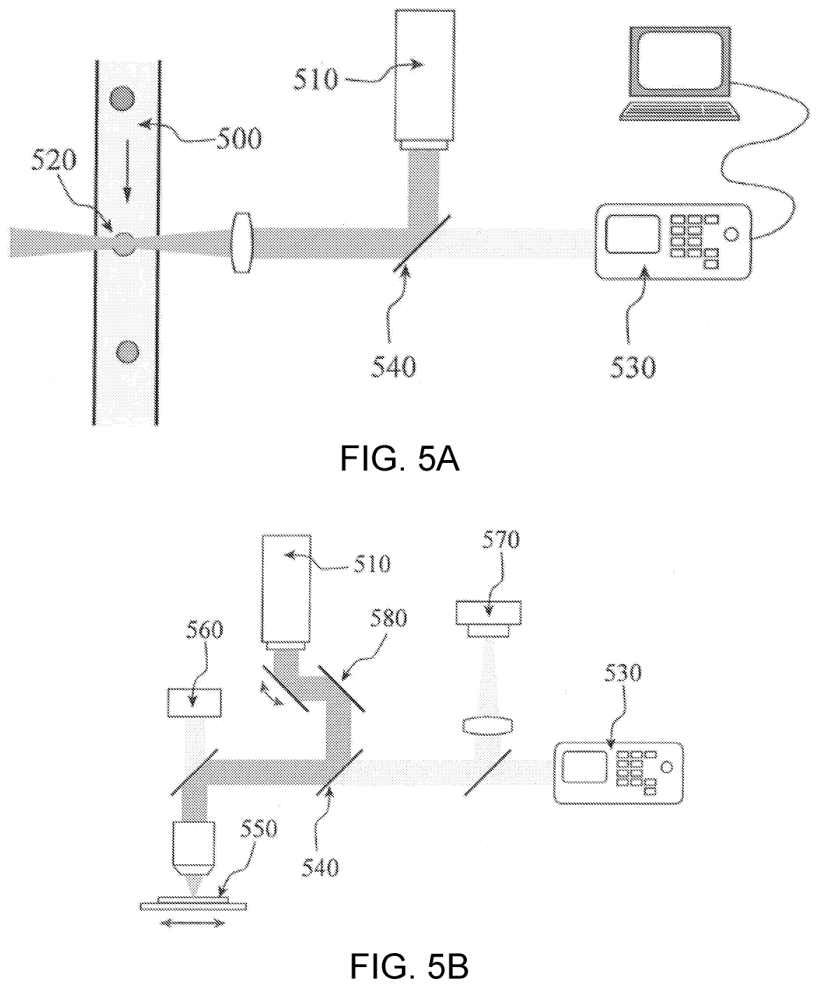

FIG. 6 shows an array of microwells 600 that may be used to capture individual cells, each of which has a fluorescent reporter, similar to that in patent publication number WO 2017/124101-A3 and Gierahn et al. Nature Methods 14(4), 395-398, 2017. The intensity of this reporter can be measured using a fluorescence microscope. Following measurement, at least one ORCP can be distributed into each microwell 610 thereby compartmentalizing individual cells with the particle. The optical barcodes of each particle 620 may then be read by an optical system similar to that shown in FIG. 5A or 5B. This procedure may allow a user to associate the fluorescent intensity of a particular reporter of a single cell 630 with the optical barcode of the resonator particle. Biomarkers of interest that are tagged with conventional fluorescent probes or reporters 640 can be read by a fluorescence microscope to determine a phenotypic property of each cell. In various embodiments, identifying a phenotypic property of a cell can include identifying an observable property related to the cell relating to at least one of protein quantification, cell cycle information, gene expression, cell location, cell mass, and intercellular interactions. In certain embodiments, identifying a phenotypic property of a cell may include directing a fluorescent excitation source at the cell; detecting fluorescent emission light from a fluorescent reporter associated with the cell based on directing the fluorescent excitation source at the cell; and identifying the phenotypic property of the cell based on detecting the fluorescent emission light. For example, tagging of α-tubulin can be used to locate and study microtubule movement during cell division, or, by tagging amyloid protein, the progress of a variety of neurodegenerative diseases can be studied, or signaling polypeptides can be tagged, allowing the study of intracellular protein trafficking.

Following the reading of both the fluorescent probe and the ORCPs' optical barcodes, the cells may then be lysed, and the contents of each individual cell may be captured by the RNA capture strands of the particular RNA capture laser particle with which the cell is compartmentalized. A cDNA library representing the captured RNA transcripts is then formed, amplified, and sequenced. Since each cDNA strand contains the cellular barcode sequence, it is possible to determine which strands originated from the same cell. Because the RNA capture particles are fabricated with a known mapping between the particle's nucleotide-based cellular barcode and its optical barcode, sequencing of the cellular barcode may also allow for association of this cellular barcode with its associated optical barcode. Since, prior to sequencing the optical barcodes may be read and associated with a particular fluorescent reporter intensity, the single cell RNA content of a particular cell can thus be associated with the phenotypic information gleaned from analysis of the fluorescent reporter.

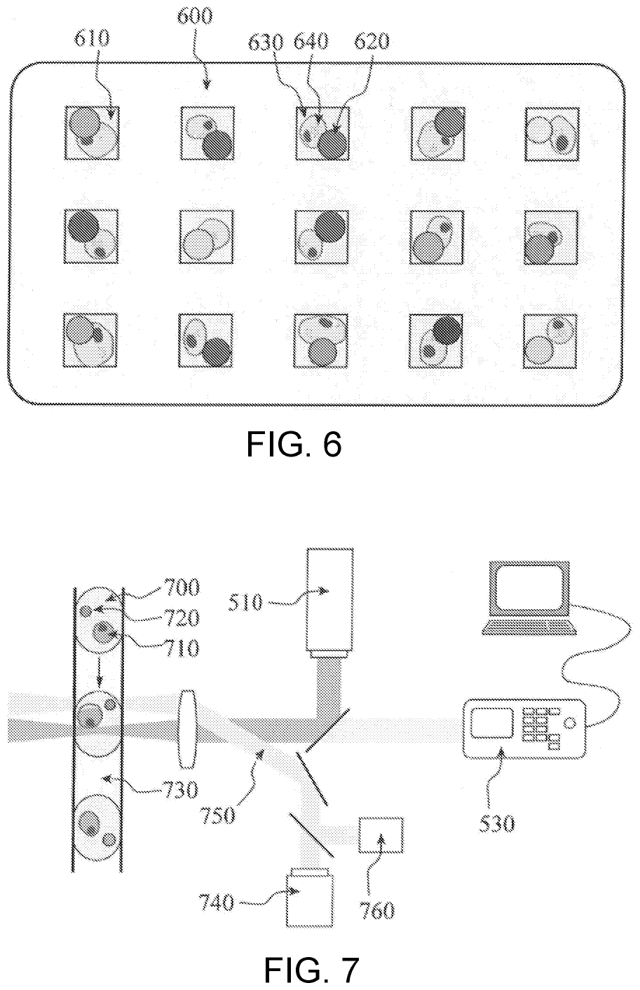

FIG. 7 shows a second embodiment of this example application. Individual aqueous-in-oil droplets 700 each containing a cell 710 and an ORCP particle 720 may be prepared, for example, using a microfluidic device. Along with a flow system containing oil 730, the optical barcode may be read using a suitable arrangement, e.g. the arrangement described in FIG. 5A. This optical barcode can then be associated with the data obtained by excitation of the fluorescent reporter or other phenotypic data obtained during this cytometric process. The fluorescent reporter on the cell may simultaneously be read by using a light source 740 emitting fluorescence excitation light 750 and a detector such as a photomultiplier tube 760. The intensity value of the fluorescent reporter corroborates a phenotypic property of the cell. Additionally, other phenotypic properties of the cell (e.g. scatter cross section, size etc.) could be analyzed and near-simultaneously coupled to single cell RNA data within a single flow system. This would allow a single device to perform both droplet-based single cell RNA sequencing as well as simultaneous analysis of phenotypic information obtained by flow cytometry.

### Example 2: High Efficiency Single Cell RNA Sequencing Using Droplet and Bead Based Methods

In droplet-based single cell sequencing methods, individual cells are encapsulated within aqueous droplets along with single RNA capture beads. However, this technique can be inefficient. For example, in a method such as Dropseq (Macosko et al. Cell 161(5), 1202-1214, 2015), the capture rate can be lower than 5% since the likelihood of a single cell being encapsulated with a single RNA capture bead follows Poisson statistics. Furthermore, if two cells are encapsulated with a single bead, the technique will incorrectly determine that the RNA from the two different cells originated from the same cell. If a single cell is encapsulated with two separate beads, the technique will incorrectly determine that the RNA from just that cell came from two separate cells.

If ORCPs are used instead of traditional RNA capture beads, the identity of the RNA capture particles that have been compartmentalized with each cell can be identified by an optical system that pumps the resonator and thus reads its optical barcode (as shown for example in FIG. 7). This can be used to eliminate the need to encapsulate a single bead with a single cell. Instead, the encapsulation condition is relaxed to allow multiple beads to be compartmentalized with a single cell, since the optical system can determine which beads were used to capture RNA from the same cell. With this relaxed condition, an excess of laser RNA capture particles could be used, so that each droplet could contain more than a single RNA capture laser particle without penalty. By using an excess of RNA capture laser particles, the encapsulation statistics approach a single Poisson distribution instead of the product of two independent Poisson distributions.

Similarly, events in which two or more cells are compartmentalized inside the same droplet can be eliminated from subsequent analysis by identifying the RNA capture resonator with which they were co-encapsulated. Events in which this is deemed to occur could be discarded from the final analysis. Therefore, theoretically, the error rate of transcripts from multiple cells being incorrectly identified as coming from just a single cell could be made to approach zero.

### Example 3: High-Throughput Spatial Transcriptomics at Cellular and Subcellular Resolution

One key deficiency in many high throughput sequencing technologies is that they do not preserve spatial information regarding the origin of the cell. The use of laser RNA capture particles can be used to overcome this deficiency. Recently, Stahl et al. (WO 2016/162309 A1 and Science 353(6294), 78-82, 2016) determined the spatial origin of RNA from fixed tissue samples by permeabilizing cells in the tissue and allowing the contained RNA to diffuse onto capture strands lining an adjacent glass slide. However, the spatial resolution of their method was low (on the order of 100 microns) blurring spatial variations in RNA that take place at the single cell level.

The use of ORCPs disclosed in this invention can be used to overcome this problem by attaching these particles to a glass slide at a density dependent on the desired sampling density. In one exemplary embodiment, this attachment might be accomplished by conjugating laser particles coated in streptavidin with a biotin coated slide.

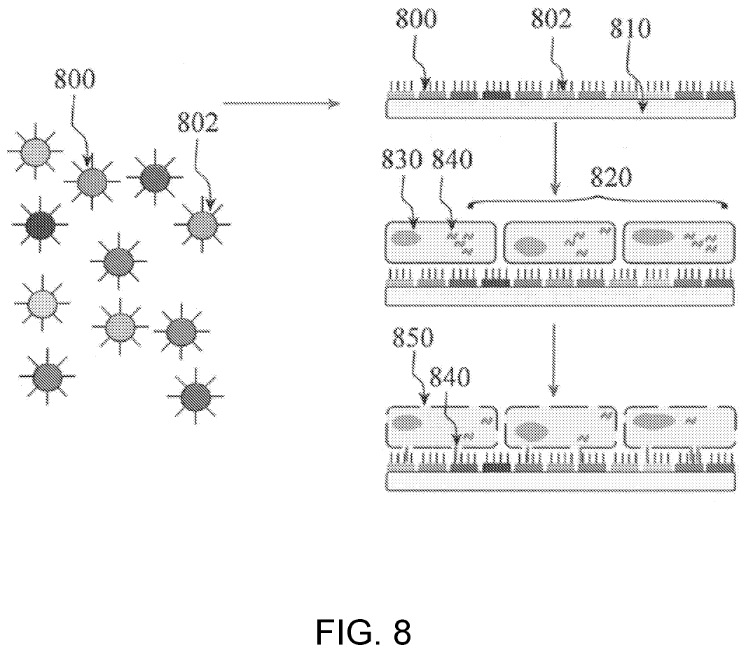

In the preferred embodiment of this invention, the ORCPs that are used may include semiconductor-based resonators. Since their size can be relatively small, a greater spatial resolution of the location of captured RNA can be obtained. FIG. 8 illustrates an exemplary embodiment of a method to determine the spatial distribution of RNA in a tissue section at a high resolution. A collection of ORCPs, such as particles 800 and 802, are deposited on a slide 810. Next, the tissue sample is sliced into thin sections (a few tens of microns, or less, in thickness) using a cryostat. Sections 820 can then be fixed and stained with hematoxylin and eosin and mounted onto the slide 810 on top of the ORCPs. Following mounting onto a microscope stage, images (e.g. bright field) are taken of the tissue section. Next, the positions of each particle, such as particle 800 or 802, are deduced by an optical system capable of pumping the particle and recording its spectral output (see, for example. FIGS. 5A, 5B). The optical barcode can thus be read and the position of each capture particle associated with a particular spatial location of the overlaying thin tissue section 820 including cells 830 that are to be analyzed may be determined. Next, permeabilization of the tissue section can be performed. This step allows the tissue's resident RNA 840 to escape and diffuse through the permeated cell membrane pores 850 towards the glass slide 810 upon which the RNA capture particles sit. Since each laser RNA capture particle contains an mRNA capture region, the RNA 840 is captured on the surface of the RNA capture laser particles. Previous work following a similar procedure (Stahl et al. Science 353(6294), 78-82, 2016) found that lateral diffusion of RNA was 1.7±2 microns (mean±standard deviation). In order to further decrease lateral diffusion, an electric field can be applied to the sample, to vertically drive the RNA molecules towards the glass slide.

Upon RNA capturing, reverse transcription reagents are then added to the slide with the attached RNA laser capture particles, generating a cDNA library. The tissue sample can then be removed, and the RNA, cDNA hybrids are cleaved from the slide for subsequent in vitro amplification and next-generation sequencing. Following sequencing of the cDNA library generated from the RNA captured on the ORCPs, the position of origin of the initially captured RNA can be deduced. This is performed by associating the cellular barcode, which was transferred to the cDNA strand during reverse transcription of the RNA oligonucleotide capture sequence, with the particle's optical barcode.

Since both the diffusion distance of the RNA released from the tissue and the size of the ORCP can be significantly smaller than the dimensions of a single cell, the method disclosed in this invention can be used to sequence RNA at a spatial resolution smaller than that of a single cell. This invention thus allows cellular and subcellular transcriptome analysis. RNA sequencing data can then be digitally overlaid with image of the tissue section to label each cell (or even each part of the cell) with the RNA transcripts that originated from that location. Such a tool would be of great use in diagnostic pathology.

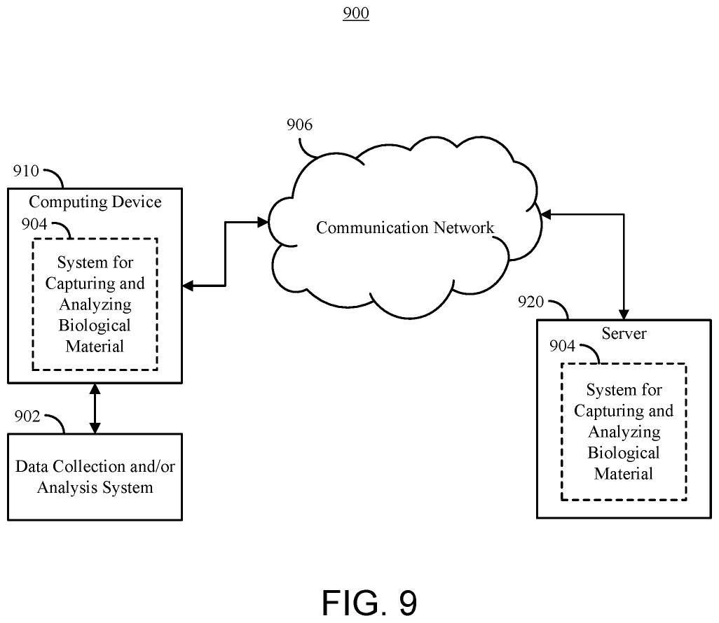

Turning to FIG. 9, an example 900 of an apparatus or system for capturing and analyzing biological material is shown in accordance with some embodiments of the disclosed subject matter. As shown in FIG. 9, a computing device 910 can receive information regarding a biological material to be captured and/or analyzed from a data collection and/or analysis system 902. In some embodiments, computing device 910 can execute at least a portion of a system for capturing and analyzing biological material 904 to identify a biological material based on the information regarding the sample received from the data collection and/or analysis system 902. Additionally or alternatively, in some embodiments, computing device 910 can communicate information about the sample received from the data collection and/or analysis system 902 to a server 920 over a communication network 906, which can execute at least a portion of system for capturing and analyzing biological material 904 to identify the biological material in the sample. In some such embodiments, server 920 can return information to computing device 910 (and/or any other suitable computing device) indicative of an output of system for capturing and analyzing biological material 904, such as an identity of the biological material in the sample. This information may be transmitted and/or presented to a user (e.g. a researcher, an operator, a clinician, etc.) and/or may be stored (e.g. as part of a research database or a medical record associated with a subject).

In some embodiments, computing device 910 and/or server 920 can be any suitable computing device or combination of devices, such as a desktop computer, a laptop computer, a smartphone, a tablet computer, a wearable computer, a server computer, a virtual machine being executed by a physical computing device, etc. As described herein, system for capturing and analyzing biological material 904 can present information indicative of an output of system for capturing and analyzing biological material 904, such as an identity of the biological material in the sample, to a user (e.g., researcher and/or physician).

In some embodiments, communication network 906 can be any suitable communication network or combination of communication networks. For example, communication network 906 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, etc.), a wired network, etc. In some embodiments, communication network 906 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, etc.

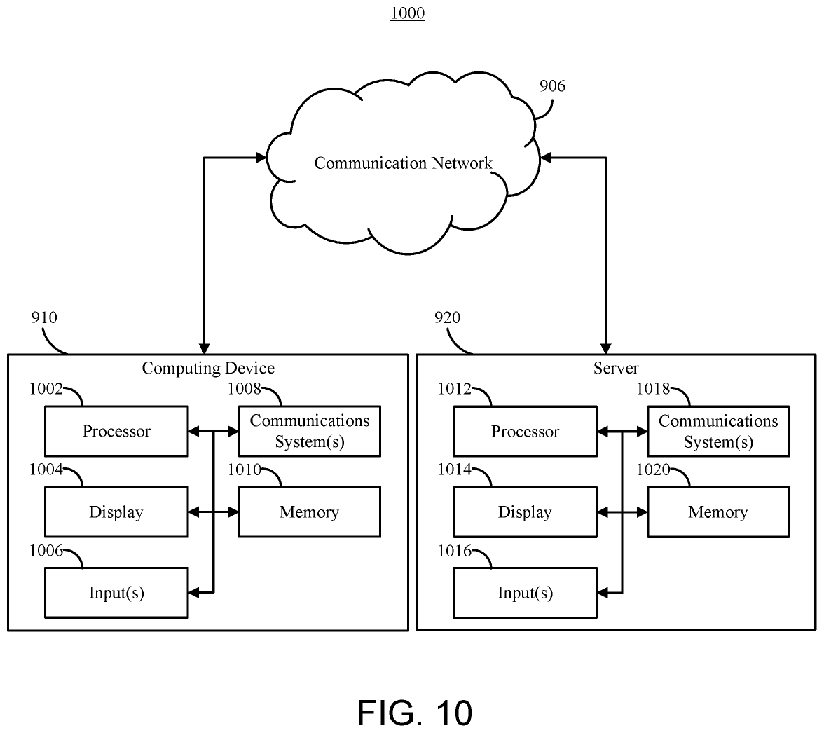

FIG. 10 shows an example 1000 of hardware that can be used to implement computing device 910 and server 920 in accordance with some embodiments of the disclosed subject matter. As shown in FIG. 10, in some embodiments, computing device 910 can include a processor 1002, a display 1004, one or more inputs 1006, one or more communication systems 1008, and/or memory 1010. In some embodiments, processor 1002 can be any suitable hardware processor or combination of processors, such as a central processing unit, a graphics processing unit, etc. In some embodiments, display 1004 can include any suitable display devices, such as a computer monitor, a touchscreen, a television, etc. In some embodiments, inputs 1006 can include any suitable input devices and/or sensors that can be used to receive user input, such as a keyboard, a mouse, a touchscreen, a microphone, etc.

In some embodiments, communications systems 1008 can include any suitable hardware, firmware, and/or software for communicating information over communication network 906 and/or any other suitable communication networks. For example, communications systems 1008 can include one or more transceivers, one or more communication chips and/or chip sets, etc. In a more particular example, communications systems 1008 can include hardware, firmware and/or software that can be used to establish a Wi-Fi connection, a Bluetooth connection, a cellular connection, an Ethernet connection, etc.

In some embodiments, memory 1010 can include any suitable storage device or devices that can be used to store instructions, values, etc., that can be used, for example, by processor 1002 to present content using display 1004, to communicate with server 920 via communications system(s) 1008, etc. Memory 1010 can include any suitable volatile memory, non-volatile memory, storage, or any suitable combination thereof. For example, memory 1010 can include RAM, ROM, EEPROM, one or more flash drives, one or more hard disks, one or more solid state drives, one or more optical drives, etc. In some embodiments, memory 1010 can have encoded thereon a computer program for controlling operation of computing device 910. In such embodiments, processor 1002 can execute at least a portion of the computer program to present content (e.g., images, user interfaces, graphics, tables, etc.), receive content from server 920, transmit information to server 920, etc.

In some embodiments, server 920 can include a processor 1012, a display 1014, one or more inputs 1016, one or more communications systems 1018, and/or memory 1020. In some embodiments, processor 1012 can be any suitable hardware processor or combination of processors, such as a central processing unit, a graphics processing unit, etc. In some embodiments, display 1014 can include any suitable display devices, such as a computer monitor, a touchscreen, a television, etc. In some embodiments, inputs 1016 can include any suitable input devices and/or sensors that can be used to receive user input, such as a keyboard, a mouse, a touchscreen, a microphone, etc.

In some embodiments, communications systems 1018 can include any suitable hardware, firmware, and/or software for communicating information over communication network 906 and/or any other suitable communication networks. For example, communications systems 1018 can include one or more transceivers, one or more communication chips and/or chip sets, etc. In a more particular example, communications systems 1018 can include hardware, firmware and/or software that can be used to establish a Wi-Fi connection, a Bluetooth connection, a cellular connection, an Ethernet connection, etc.

In some embodiments, memory 1020 can include any suitable storage device or devices that can be used to store instructions, values, etc., that can be used, for example, by processor 1012 to present content using display 1014, to communicate with one or more computing devices 910, etc. Memory 1020 can include any suitable volatile memory, non-volatile memory, storage, or any suitable combination thereof. For example, memory 1020 can include RAM, ROM, EEPROM, one or more flash drives, one or more hard disks, one or more solid state drives, one or more optical drives, etc. In some embodiments, memory 1020 can have encoded thereon a server program for controlling operation of server 920. In such embodiments, processor 1012 can execute at least a portion of the server program to transmit information and/or content (e.g., information regarding the virtual lens, the desired intensity pattern, the phase mask, any data collected from a sample that is illuminated, a user interface, etc.) to one or more computing devices 910, receive information and/or content from one or more computing devices 910, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The apparatus and methods disclosed herein are not limited to the capture of RNA from single cells. In another embodiment, ‘accessible chromatin in single cells using sequencing’ (scATAC-seq) (Sapathy et al. Nature Biotechnology 37, 925-936 (2019)) is used with ORCPs to measure chromatin expression and associate that with some cellular/nuclear phenotypic property. chromatin is instead captured by the capture sites of the ORCP. This can be accomplished by isolating cells or cell nuclei from a cell suspension and performing a bulk transposition with transposase Tn5. This enzyme cuts and ligates adapter sequences to the nuclear chromatin, in open, accessible regions of DNA. The single cells/nuclei are then isolated with ORCPs, for example, in a water-in-oil droplets (FIG. 7) or in microwells (FIG. 6). The DNA fragments generated by transposition are then integrated into the DNA barcodes on the ORCP, thus preparing a library ready for sequencing. Phenotypic properties of the cell/nuclei can be read following their isolation with a particular ORCP as well as the ORCP's optical barcode. The generation of chromatin sequencing data can then be mapped to any phenotypic measurement through the known association of the ORCP's cellular barcode with its optical barcode.

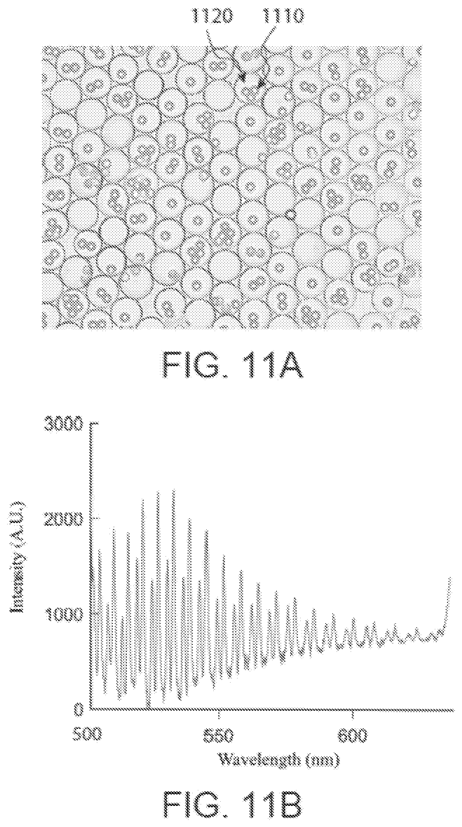

FIG. 11A shows an exemplary embodiment of an ORCP comprising a polyacrylamide hydrogel bead coupled to RNA capture strands. To create the optical barcode, a plurality of polystyrene microspheres 1110, doped with a fluorescent gain material, of size approximately 10 μm diameter are embedded inside a hydrogel bead 1120. Since the refractive index of the hydrogel (even at a low water content) is less than that of the polystyrene microspheres, we see distinct spectral peaks at the resonant wavelengths of the microsphere. These ORCPs are formed using water-in-oil emulsions in which the aqueous phase contains a suspension of the polystyrene microspheres. A sample spectral emission is shown in FIG. 11B, showing the peaks that contribute to the optical barcode. The number of uniquely identifiable optical barcodes increases with the number of polystyrene microspheres (ORPs) in each hydrogel ORCP. We can estimate the number of uniquely identifiable barcodes using combinatorics, as ^{n×g}ₘC where: n is the number of uniquely identifiable spectra if only a single microsphere were used, which has been estimated as 2,000 for a set of microspheres between 8 μm and 12 μm (Humar et al. Nature Photonics. 2015, 9(9) 572-576); g is the number of gain media; and m is the number of microspheres within the ORCP. Therefore, with three microspheres and 5 different gain media, for example, the number of uniquely identifiable barcodes is expected to exceed many millions and is in fact approximated as ^{10,000}₅C=˜10¹¹.

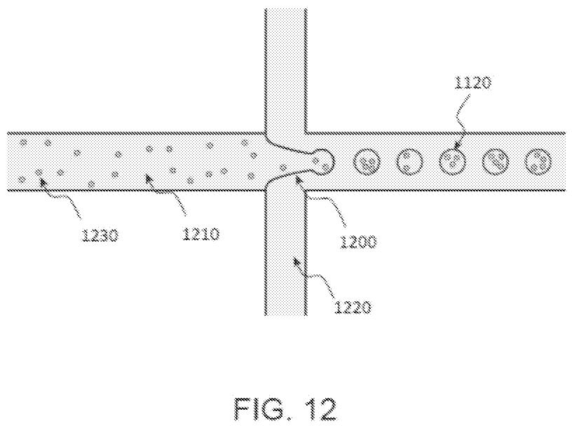

FIG. 12 shows an embodiment of a microfluidic process that may be used to form the ORCP. A microfluidic flow-focusing junction 1200 forms the droplets, which are stabilized using an appropriate surfactant. The aqueous phase containing the acrylamide precursors and microspheres is shown by 1210 and the oil phase (which may itself contain activators that help form the hydrogel ORCPs) is shown by 1220. To prevent microsphere aggregation, the surface of the microspheres (1230) is functionalized with carboxylate groups. Furthermore, the aqueous solution contains precursor material for forming the hydrogel microspheres (in this case acrylamide), which are recovered from the microfluidic device and heat cured. Since droplet encapsulation is a stochastic process, some hydrogel beads remain empty, even when a high concentration of polystyrene microspheres are used. Fluorescence based sorting techniques can be used to remove these empty beads if necessary. Depending on the number of uniquely identifiable optical barcodes, the number of barcoding polystyrene microspheres (ORPs) may need to be more than 3, for example; then ORCPs that include 0, 1, or 2 ORPs and thus are likely to have non-unique barcodes may be discarded if necessary.

### REFERENCES

Each of the following references is incorporated herein by reference in its entirety:
Regev et al. A droplet-based method and apparatus for composite single-cell nucleic acid analysis. WO 2016040476 A1.
Weitz et al. Systems and methods for barcoding nucleic acids. WO 2015164212 A1.
Gierahn et al. Semi-permeable arrays for analyzing biological systems and methods of using same. WO 2017124101 A3.
Frisen et al. Spatially distinguished, multiplex nucleic acid analysis of biological specimens. WO 2016162309 A1.
Single-cell barcoding and sequencing using droplet microfluidics. Zilionis et al. Nature Protocols 12, 44-73, 2017.
Seq-Well: portable, low-cost RNA sequencing of single cells at high throughput. Gierahn et al. Nature Methods, 14(4), 395-398, 2017.
Highly Parallel Genome-wide Expression Profiling of Individual Cells Using Nanoliter Droplets. Macosko et al. Cell. 161(5), 1202-1214, 2015.
Visualization and analysis of gene expression in tissue sections by spatial transcriptomics. Stahl et al. Science. 353(6294), 78-82, 2016.
Spectral reading of optical resonance-encoded cells in microfluidics. Humar et al. Lab Chip, 2017, 17, 2777.
Geometrical theory of whispering-gallery modes. Gorodetsky and Fomin. IEEE J. Sel. Topics Quantum Electron. 12, 33-39, 2006.
Massively parallel single-cell chromatin landscapes of human immune cell development and intratumoral T cell exhaustion. Sapathy et al. Nature Biotechnology 37, 925-936 (2019).
Intracellular microlasers. Humar et al. Nature Photonics. 2015, 9(9) 572-576.

It will be apparent to those skilled in the art that numerous changes and modifications can be made in the specific embodiments of the invention described above without departing from the scope of the invention. Accordingly, the whole of the foregoing description is to be interpreted in an illustrative and not in a limitative sense.

1. An apparatus for capturing biological material, comprising:
an optically readable capture particle (ORCP) comprising:
one or more optically readable particles (ORPs) each comprising an optical barcode to identify the ORCP; and
a plurality of biological capture sites associated with the one or more ORPs,
each of the plurality of biological capture sites including a cellular barcode to identify the ORCP.

2. The apparatus of claim 1, wherein the one or more ORPs comprise a resonator and gain medium, wherein the gain medium comprises a fluorescent material.

3. The apparatus of claim 2, wherein the one or more ORPs comprise a microsphere, wherein the microsphere is doped with the gain medium.

4-5. (canceled)

6. The apparatus of claim 2, wherein the fluorescent material is at least one of a fluorescent dye, a quantum dot, or a protein.

7. The apparatus of claim 3, wherein the microsphere comprises a polystyrene microsphere.

8. The apparatus of claim 3, wherein the microsphere has a diameter of at least 3 μm.

9. The apparatus of claim 1, wherein the optical barcode comprises an emission spectrum having at least one peak.

10. The apparatus of claim 1, wherein the ORCP comprises a plurality of gain media and a plurality of resonators,
wherein each of the plurality of gain media comprises a different emission spectrum.

11. The apparatus of claim 2, wherein the one or more ORPs comprise a semiconductor particle which comprises the resonator and the gain medium.

12. The apparatus of claim 11, wherein the semiconductor particle is contained within a transparent coating.

13. The apparatus of claim 12, wherein the one or more ORPs comprise a plurality of semiconductor particles contained within the transparent coating, wherein each of the plurality of semiconductor particles comprises a gain medium having a different emission spectrum from the other of the plurality of semiconductor particles.

14. (canceled)

15. The apparatus of claim 1, wherein each of the plurality of biological capture sites comprises a nucleotide strand configured to capture RNA wherein the nucleotide strand comprises an oligonucleotide cellular barcode sequence.

16. (canceled)

17. A method of capturing biological material, comprising:
combining an ORCP and a cell within an aqueous environment, the ORCP comprising:
one or more optically readable particles (ORPs) each comprising an optical barcode to identify the ORCP, and
a plurality of biological capture sites coupled with the one or more ORPs,
each of the plurality of biological capture sites including a cellular barcode to identify the ORCP, and
each of the plurality of biological capture sites comprising a capture site for capture of biological material;
reading the optical barcode of the ORCP;
identifying a phenotypic property of the cell;
capturing contents of the cell such that they interact with the plurality of biological capture sites of the ORCP; and
processing the ORCP to identify the contents of the cell associated with the plurality of biological capture sites.

18. The method of claim 17, wherein the one or more ORPs comprise a resonator and a gain medium.

19. The method of claim 18, wherein the one or more ORPs comprise a microsphere, wherein the microsphere is doped with the gain medium.

20. (canceled)

21. The method of claim 18, wherein the gain medium comprises a fluorescent material comprising at least one of a fluorescent dye, a quantum dot, or a protein.

22. The method of claim 19, wherein the microsphere comprises a polystyrene microsphere having a diameter of at least 3 μm.

23. The method of claim 18, wherein the one or more ORCP comprise a semiconductor particle.

24. The method of claim 23, wherein the semiconductor particle comprises the resonator and the gain medium.

25. The method of claim 23, wherein the semiconductor particle is contained within a transparent coating.

26. The method of claim 25, wherein the ORCP comprises a plurality of semiconductor particles contained within the transparent coating, wherein each of the plurality of semiconductor particles comprises a gain medium having a different emission spectrum from the other of the plurality of semiconductor particles.

27. (canceled)

28. The method of claim 17, wherein the optical barcode comprises an emission spectrum having at least one peak.

29. The method of claim 1, wherein each of the plurality of biological capture sites comprises a nucleotide strand configured to capture RNA wherein the nucleotide strand comprises an oligonucleotide cellular barcode sequence.

30-32. (canceled)

33. The method of claim 17, wherein reading the optical barcode of the ORCP further comprises:
directing a light source at the ORCP;
detecting a return light spectrum emitted by the ORCP based on directing the light source at the ORCP; and
determining the optical barcode by analyzing the return light spectrum to identify at least one peak within the return light spectrum.

34. The method of claim 17, wherein identifying a phenotypic property of the cell further comprises:
identifying an observable property related to the cell relating to at least one of protein quantification, cell cycle information, gene expression, cell location, cell mass, and intercellular interactions.

35. The method of claim 17, wherein identifying a phenotypic property of the cell further comprises:
directing a fluorescent excitation source at the cell;
detecting fluorescent emission light from a fluorescent reporter associated with the cell based on directing the fluorescent excitation source at the cell; and
identifying the phenotypic property of the cell based on detecting the fluorescent emission light.

36. The method of claim 17, wherein combining an ORCP and a cell within an aqueous environment further comprises:
providing a plurality of ORCPs on a surface;
placing the cell adjacent the plurality of ORCPs;
identifying a location of each of the plurality of ORCPs relative to the cell by reading the optical barcode of each of the plurality of ORCPs;
identifying the phenotypic property of the cell;
releasing cellular contents from the cell; and
processing each of the plurality of ORCPs to identify the contents of the cell associated with the plurality of biological capture sites associated with each of the plurality of ORCPs.

37-43. (canceled)

44. An apparatus for capturing biological material including an optically readable capture particle (ORCP) comprising:
a plurality of optically readable particles (ORPs) and a plurality of oligonucleotide-based cellular barcodes in which an association has been established between an optical barcode of the plurality of ORPs and the oligonucleotide-based cellular barcode, wherein:
knowledge of a sequence of the oligonucleotide-based cellular barcode enables a determination of the optical barcode of the plurality of ORPs, or
knowledge of the optical barcode of the plurality of ORPs enables a determination of the oligonucleotide-based cellular barcode.
